(19) United States
(12) Patent Application Publication
(10) Pub. No.: US 2016/0206216 A1
(43) Pub. Date: Jul. 21, 2016

(54) DEVICE, SYSTEM AND METHOD FOR SKIN DETECTION

(71) Applicant: KONINKLIJKE PHILIPS N.V., EINDHOVEN (NL)

(72) Inventor: Ihor Olehovych KIRENKO, Veldhoven (NL)

(21) Appl. No.: 14/995,626

(22) Filed: Jan. 14, 2016

Related U.S. Application Data
(60) Provisional application No. 62/104,953, filed on Jan. 19, 2015.

(30) Foreign Application Priority Data
Jan. 19, 2015 (EP) 15151573.1

(51) Int. Cl. A61B 5/0205

(52) U.S. Cl. CPC A61B 5/02055; A61B 5/015

(57) ABSTRACT

A device, system and method for skin detection are presented. To enable a reliable, accurate and fast detection the proposed device comprises a thermal sensor input for obtaining thermal sensor data of a scene, a light sensor input for obtaining light sensor data of the scene, and an evaluation unit for analyzing the obtained thermal sensor data and the obtained light sensor data and for detecting skin areas within the scene based on said analysis.

### CROSS REFERENCE TO RELATED APPLICATIONS

This application claims the benefit of European Patent Application Number 15151573.1 filed Jan. 19, 2015 and claims the benefit of U.S. Provisional Patent Application No. 62/104,953 filed Jan. 19, 2015. These applications are hereby incorporated by reference herein.

### FIELD OF THE INVENTION

The present invention relates to a device, system and method for skin detection.

### BACKGROUND OF THE INVENTION

Vital signs of a person, for example the heart rate (HR), the respiration rate (RR) or the arterial blood oxygen saturation (SpO2), serve as indicators of the current health state of a person and as powerful predictors of serious medical events. For this reason, vital signs are extensively monitored in inpatient and outpatient care settings, at home or in further health, leisure and fitness settings.

One way of measuring vital signs is plethysmography. Plethysmography generally refers to the measurement of volume changes of an organ or a body part and in particular to the detection of volume changes due to a cardio-vascular pulse wave traveling through the body of a subject with every heartbeat.

Photoplethysmography (PPG) is an optical measurement technique that evaluates a time-variant change of light reflectance or transmission of an area or volume of interest. PPG is based on the principle that blood absorbs light more than surrounding tissue, so variations in blood volume with every heart beat affect transmission or reflectance correspondingly. Besides information about the heart rate, a PPG waveform can comprise information attributable to further physiological phenomena such as the respiration. By evaluating the transmittance and/or reflectivity at different wavelengths (typically red and infrared), the blood oxygen saturation can be determined.

Recently, non-contact, remote PPG (rPPG) devices (also called camera rPPG devices) for unobtrusive measurements have been introduced. Remote PPG utilizes light sources or, in general radiation sources, disposed remotely from the subject of interest. Similarly, also a detector, e.g., a camera or a photo detector, can be disposed remotely from the subject of interest. Therefore, remote photoplethysmographic systems and devices are considered unobtrusive and well suited for medical as well as non-medical everyday applications. This technology particularly has distinct advantages for patients with extreme skin sensitivity requiring vital signs monitoring such as Neonatal Intensive Care Unit (NICU) patients with extremely fragile skin or premature babies.

Verkruysse et al., “Remote plethysmographic imaging using ambient light”, Optics Express, 16(26), 22 Dec. 2008, pp. 21434-21445 demonstrates that photoplethysmographic signals can be measured remotely using ambient light and a conventional consumer level video camera, using red, green and blue color channels.

Wieringa, et al., “Contactless Multiple Wavelength Photoplethysmographic Imaging: A First Step Toward “SpO2 Camera” Technology,” Ann. Biomed. Eng. 33, 1034-1041 (2005), discloses a remote PPG system for contactless imaging of arterial oxygen saturation in tissue based upon the measurement of plethysmographic signals at different wavelengths. The system comprises a monochrome CMOS-camera and a light source with LEDs of three different wavelengths. The camera sequentially acquires three movies of the subject at the three different wavelengths. The pulse rate can be determined from a movie at a single wavelength, whereas at least two movies at different wavelengths are required for determining the oxygen saturation. The measurements are performed in a darkroom, using only one wavelength at a time.

Apart from the advantage of being fully contactless, cameras (generally called imaging devices) provide 2D information, which allows for a multi-spot and large area measurement, and often contains additional context information. Unlike with contact sensors, which rely on the correct placement on a specific measurement point/area, the regions used to measure pulse signals using rPPG technology are determined from the actual image. Therefore, accurate detection of skin areas, reliable under any illumination conditions becomes a crucial part in the processing chain of a camera-based rPPG device and method.

Currently, there are two main approaches known for reliable detection and tracking of a skin areas.

One approach is based on skin color (RGB-based) detection and segmentation. Methods according to this approach are fast in both detection and tracking of areas with skin color. However, they are not robust to changes of ambient light color, which will change the color of light reflected from a skin area, and are not able to detect skin areas under low illumination conditions or in darkness. Moreover, such methods cannot always differentiate a skin from other objects with the same color.

Another approach is based on extracted PPG signals (PPG-based). Methods according to this approach are more robust in differentiating real skin areas and areas of other object of the same skin color. This approach can be used also to segment the skin areas, which have stronger PPG signal (the most periodic signal). However, the reliability of the approach depends on the robustness of PPG signal extractions, thus it is impacted by motion of a subject and the blood perfusion level. Therefore, if a pulse signal is not periodic or weak, a camera-based system will have difficulties to detect the segment the skin areas. Moreover, the approach is also computationally expensive.

It should be noted that the detection of skin area is not only of interest in the field of vital signs detection based on the rPPG technology, but also in other technical fields, e.g. in remote gaming applications using camera technology to recognize gestures of the player, face detection, security (robust detection of a person using surveillance cameras and detection of a person wearing a mask or distinguishing real faces from a realistic mask in a camera registration), etc.

WO 2014/012070 A1 discloses an infant monitoring system including an infrared imaging module, a visible light camera, a processor, a display, a communication module, and a memory. The monitoring system may capture thermal images of a scene including at least a partial view of an infant, using the infrared imaging module enclosed in a portable or mountable housing configured to be positioned for remote monitoring of the infant. Various thermal image processing and analysis operations may be performed on the thermal images to generate monitoring information relating to the infant. The monitoring information may include various alarms that actively provide warnings to caregivers, and user-viewable images of the scene. The monitoring information may be presented at external devices or the display located remotely for convenient viewing by caregivers.

US 2013/0215928 A1 discloses a method for determining a body temperature of a patient or subject, in which a contactless infrared thermometer is manually positioned such as to point at a target area, and by activating the thermometer such that it measures the internal temperature of a patient and reads, via the thermometer, and for a predetermined time interval, the intensity of an infrared radiation in arrival from the target area. The target area is a body surface of a human or animal patient selected from a body location group comprising the ethmoid sinus, the canthus, the open eyelid or the closed eyelid.

### SUMMARY OF THE INVENTION

It is an object of the present invention to provide a device and a corresponding method as well as a system which allow a reliable, accurate and fast detection of skin, in particular for use in a device and method for detecting vital signs of a subject.

In a first aspect of the present invention a device for skin detection is presented comprising

a thermal sensor input configured to obtain thermal sensor data of a scene,

a light sensor input configured to obtain light sensor data of the scene, and

an evaluation unit configured to analyze the obtained thermal sensor data and the obtained light sensor data and to detect skin areas within the scene based on said analysis, wherein said evaluation unit is configured

to select first spatial areas within the thermal sensor data having data values in a predetermined range,

to select second spatial areas within the light sensor data having data values in a predetermined range,

to select third and/or fourth spatial areas by determining photoplethysmography, PPG, signals from the light sensor data and selecting third and/or fourth spatial areas within the light sensor data having the highest PPG signal strengths and/or having the strongest pulsatility, and/or to select fifth spatial areas within the light sensor data having the highest spatial uniformity of chrominance and/or intensity values of adjacent pixels, and

to correlate said first and second spatial areas with one or more of said third, fourth and fifth spatial areas to detect skin areas.

In a further aspect of the present invention a corresponding method is presented.

In a yet further aspect of the present invention a system is presented comprising

a thermal sensor configured to acquire thermal sensor data of a scene,

a light sensor configured to acquire light sensor data of the scene, and

a device as disclosed herein for skin detection within the scene based on the acquired thermal sensor data and the acquired light sensor data.

In yet further aspects of the present invention, there are provided a computer program which comprises comprising program code means for causing a computer to carry out the steps of the method as disclosed herein when said computer program is carried out by the computer as well as a non-transitory computer-readable recording medium that stores therein a computer program product, which, when executed by a computer, causes the method disclosed herein to be performed.

Preferred embodiments of the invention are defined in the dependent claims. It shall be understood that the claimed method, system, computer program and medium have similar and/or identical preferred embodiments as the claimed device and as defined in the dependent claims.

The present invention makes use of thermal camera technology, in particular in long-wave infrared camera technology. Nowadays, compact thermal cameras are available at a consumer price level, such as the Lepton camera module of FLIR Systems Inc., which can e.g. be integrated with consumer mobile devices. Although such consumer-level thermal cameras cannot provide the same spatial and thermal resolution as a professional camera, their performance would be sufficient to differentiate warm and cold objects. Moreover, thermal camera can also provide information about the spatial distribution of temperature, which can be used for selection of areas with expected higher level of PPG pulsatility due do the relationship between changes of skin temperature and changes of local blood flow.

The present invention provides a device, method and system of combining thermal imaging, preferably by use of a low-cost thermal sensor (preferably providing two-dimensional thermal imaging data as thermal sensor data), and RGB or IR (infrared) imaging for robust detection and preferably tracking of one or more skin detections (also called regions of interest, ROI), particularly for extraction of PPG signals from which one or more vital signs may be derived. Since a thermal sensor would detect any warm object (i.e. not only skin), a robust skin detection cannot rely on thermal imaging only. Therefore, the present invention proposes a unique method to combine thermal imaging with RGB (and/or IR)-based and/or PPG-based techniques for robust selection of skin ROI and, in a preferred embodiment, for segmentation of ROI into areas with likely high PPG pulsatility.

Said evaluation unit is configured to select first spatial areas within the thermal sensor data having data values in a predetermined range, to select second spatial areas within the light sensor data having data values in a predetermined range and to correlate said first and second spatial areas to detect skin areas. Preferably, said evaluation unit is configured to select first spatial areas within the thermal sensor data having data values in a range of normal temperature of a human skin (in particular in a range of 30 to 42° C. and to select second spatial areas within the light sensor data having data values in a range typical for skin. Thus, the detection of skin area(s) is based on the data directly acquired by the respective sensors. The range of the data values of the light sensor data typical for skin can e.g. be defined for a specific color space, in which the light sensor is operating. For instance, for a RGB sensor, the skin color range would be 20 points difference between red (R) and blue (B) with green (GG as close to the middle as possible (e.g. 75% R, 65% G, 55% B). For darker skin, the numbers will be higher, for more yellow skin there will be a decreased blue value, etc. For other color spaces (e.g. YUV), the skin color range would be defined in a similar way, i.e. as a combination of thresholds in each of the channels.

In another option said evaluation unit is configured to select first spatial areas within the thermal sensor data having data values in a predetermined range, to determine photoplethysmography, PPG, signals from the light sensor data, to select third spatial areas within the light sensor data having the highest PPG signal strengths and to correlate said first and third spatial areas to detect skin areas. Thus, the detection of skin area(s) is not only based on the data directly acquired by the respective sensors, but also on data derived there from, namely PPG signals and the respective signal strengths of PPG signals, which is particularly useful, if vital signs shall be obtained from the detected skin area(s).

In still another option said evaluation unit is configured to select first spatial areas within the thermal sensor data having data values in a predetermined range, to determine photoplethysmography, PPG, signals from the light sensor data, to select fourth spatial areas within the light sensor data having the strongest pulsatility and to correlate said first and fourth spatial areas to detect skin areas. Also in this embodiment the detection of skin area(s) is not only based on the data directly acquired by the respective sensors, but also on data derived there from, namely PPG signals and the respective pulsatility of PPG signals, which is particularly useful, if vital signs shall be obtained from the detected skin area(s).

In still another option said evaluation unit is configured to select first spatial areas within the thermal sensor data having data values in a predetermined range, to select fifth spatial areas within the light sensor data having the highest spatial uniformity of chrominance and/or intensity values of adjacent pixels and to correlate said first and fifth spatial areas to detect skin areas. Again, in this embodiment the detection of skin area(s) is not only based on the data directly acquired by the respective sensors, but also on data derived there from, namely based on the uniformity of chrominance and/or intensity values.

According to embodiments of the present invention, not only two spatial areas are correlated, but further spatial areas selected in different ways may be correlated to further increase the reliability and robustness of skin area detection. Accordingly, said evaluation unit is configured to select second spatial areas within the light sensor data having data values in a predetermined range and to correlate said first and second spatial areas with one or more of said third, fourth and fifth spatial areas to detect skin areas.

The skin area detection may be applied in various applications. A preferred application is the use in the detection of vital signs of a subject, in particular a person. Accordingly, in an embodiment the device further comprises a vital signs detector for detecting vital signs of a subject within the scene based on image information from detected skin areas within said sequence of images. The vital signs are preferably obtained by use of the known remote PPG technology or from motion of skin areas caused by pulse. Respiratory information, e.g. the respiration rate, may also be obtained from movements of the skin, e.g. the chest wall or the belly area caused by respiration.

Still further, in an embodiment said evaluation unit is configured to spatially align the obtained thermal sensor data and the obtained light sensor data before the analysis. This alignment may e.g. be performed digitally by use of image processing techniques, such as known registration algorithms. This embodiment particularly enables supplementing an existing device, e.g. for obtaining vital signs of a subject by use of remotely acquired image data from a camera, with a thermal sensor to increase the robustness of skin detection from which vital signs are then derived by use of remote PPG technology.

Various embodiments exist for obtaining the thermal sensor data and the light sensor data. In a practical realization said thermal sensor comprises a longwave camera unit for acquiring thermal images in the longwave infrared spectrum and said light sensor comprises an imaging unit, e.g. a camera, for acquiring images in the visible and/or infrared light spectrum.

The invention is preferably used in the context of vital signs acquisition by use of the remote PPG technology. For this purpose said imaging unit is preferably configured to acquire a sequence of images of the scene over time, and said device further comprises a vital signs detector for detecting vital signs of a subject within the scene based on image information from detected skin areas within said sequence of images. Thus, the proposed detection of skin areas may be once or continuously used to detect and/or track skin areas during the acquisition of vital signs.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a first embodiment of a system and a device according to the present invention,

FIG. 2 shows a schematic diagram of a second embodiment of a device according to the present invention,

FIG. 3 shows a schematic diagram of a third embodiment of a device according to the present invention, and

FIG. 4 shows a flow chart of an embodiment of a method according to the present invention.

### DETAILED DESCRIPTION OF THE INVENTION

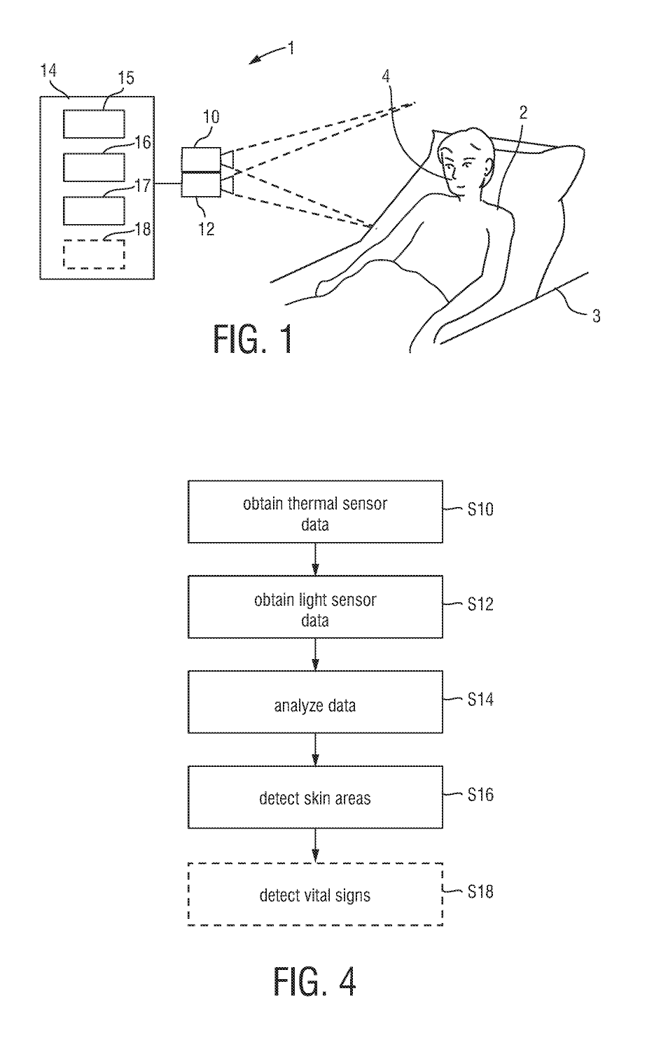

FIG. 1 shows a schematic diagram of a first embodiment of a system 1 including a device 14 for skin detection according to the present invention. The system 1 comprises a thermal sensor 10 for acquiring thermal sensor data of a scene. The scene includes, in this example, a patient 2 lying in a bed 3, e.g. in a hospital room or other healthcare facility, but may also be the environment of a neonate or premature infant, e.g. lying in an incubator/warmer, or a person at home or in a different environment. The system 1 further comprises a light sensor 12 for acquiring light sensor data of the scene. Still further, the system 1 comprises a device 14 for skin detection within the scene based on the acquired thermal sensor data and the acquired light sensor data.

The thermal sensor 10 may be a longwave infrared camera for acquiring longwave images (also called thermograms), as for instance used in infrared thermography, thermal imaging and thermal video. Such a thermal sensor 10 particularly detects radiation in the infrared range of the electromagnetic spectrum (roughly 7 to 14 μm) and produces images of that radiation.

The light sensor 12 may be an imaging unit for acquiring an image (also called RGB image hereinafter, which shall be understood as an image in the wavelength range of visual and/or infrared light) of the scene. The imaging unit is particularly a camera (also referred to as detection unit or as camera-based or remote PPG sensor), which is configured to obtain images of the scene, preferably including skin areas 4 of the patient 2. The light sensor 12 can be a part of a thermal sensor 10 or a separate device/unit. In an application of the device for obtaining vital signs of the patient 2, the skin area 4 is preferably an area of the face, such as the cheeks or the forehead, but may also be another area of the body with visible skin surface, such as the hands or the arms.

The image frames captured by the imaging may particularly correspond to a video sequence captured by means of an analog or digital photosensor, e.g. in a (digital) camera. Such a camera usually includes a photosensor, such as a CMOS or CCD sensor, which may also operate in a specific spectral range (visible, nIR) or provide information for different spectral ranges, particularly enabling the extraction of PPG signals. The camera may provide an analog or digital signal. The image frames include a plurality of image pixels having associated pixel values. Particularly, the image frames include pixels representing light intensity values captured with different photosensitive elements of a photosensor. These photosensitive elements may be sensitive in a specific spectral range (i.e. representing a specific color). The image frames include at least some image pixels being representative of a skin portion of the person. Thereby, an image pixel may correspond to one photosensitive element of a photo-detector and its (analog or digital) output or may be determined based on a combination (e.g. through binning) of a plurality of the photosensitive elements. Similarly, the thermal images obtained by the thermal camera may be a time sequence of thermal images captured over time, preferably in parallel to the image acquisition of the imaging unit.

The thermal sensor 10 and the light sensor 12 are preferably integrated into a common device, preferably such that they have substantially the same optical path. A single optical path guarantees that the pixels in a thermal images and visual light images are spatially aligned. In other embodiments the thermal sensor 10 and the light sensor 12 are implemented as separate units and the alignment of the obtained data is achieved by image processing techniques.

The device 14 comprises a thermal sensor input 15 for obtaining thermal sensor data of the scene, a light sensor input 16 for obtaining light sensor data of the scene, and an evaluation unit 17 for analyzing the obtained thermal sensor data and the obtained light sensor data and for detecting skin areas within the scene based on said analysis. Preferably, the thermal sensor data are directly obtained from the thermal sensor 10 and the light sensor data are directly obtained from the light sensor 12, and also the detection of the skin area(s) is performed on the fly. However, in other embodiments the thermal sensor data and the light sensor data as acquired by the respective sensor may be buffered for later use and evaluation, so that the device 14 obtains from a buffer or other storage medium.

The device 14 may further comprise a controller for controlling the other elements of the system 1 and a user interface, such as a keyboard and/or a display, for entering commands for controlling the device 1 and/or outputting generated information, such as obtained vital signs. Still further, the device 14 preferably comprises a vital signs detector 18 for detecting vital signs of a subject within the scene based on image information from detected skin areas within the sequence of images acquired by the light sensor 12.

The units 15 to 18 may be configured as dedicated hardware elements, but may also be configured as processor or computer, which is programmed accordingly. The device 14 may be configured as integrated device including all its elements and units, e.g. in a common housing (e.g. in a common housing of the thermal sensor 10 and the light sensor 12) or as distributed device, as shown in FIG. 1, in which the elements and units may be distributed, i.e. implemented as separate elements and units arranged at different positions.

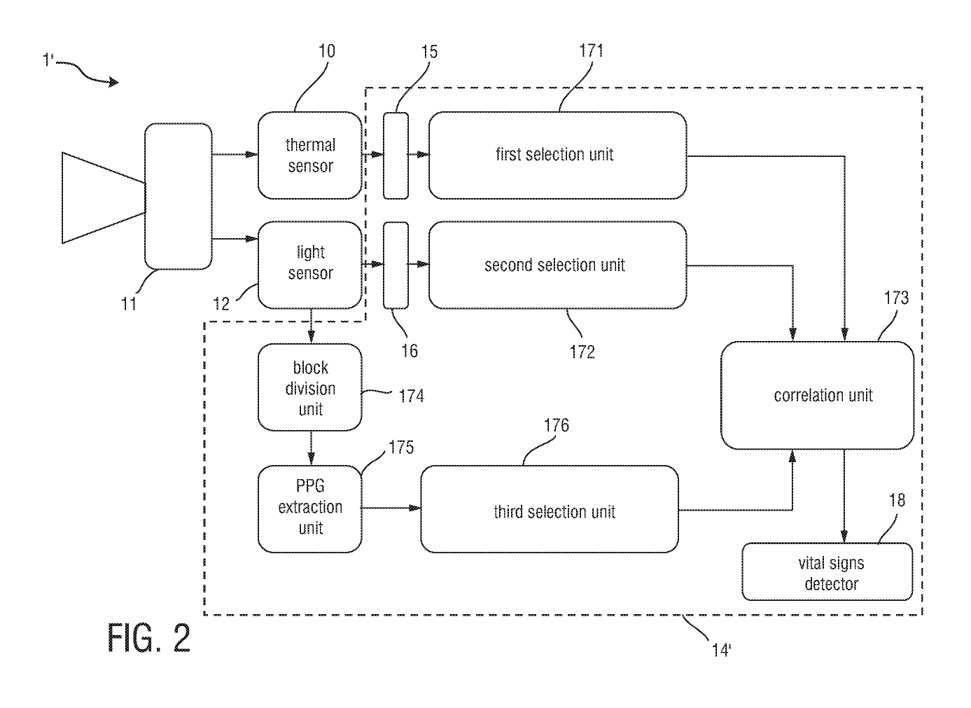

A schematic diagram of another embodiment of the system 1′ and the device 14′ according to the present invention is shown on FIG. 2. The thermal sensor 10 and the light sensor 12 obtain data from a (common) camera 11. Pixels with values, which correspond to a certain range of temperature, are selected on the thermal image by a first selection unit 171. For instance, this range can be between 30 and 42° C. The selected pixels will correspond to not only skin areas, but also to other surfaces with the same temperature. For instance, pixels on a shirt of a person would substantially have the same temperature values as skin pixels. Therefore, selection of skin pixels based only on thermal imaging is not sufficiently reliable.

A second selection unit 172 processes images obtained from the light sensor 12 to select light pixels (e.g. RGB pixels and/or infrared pixels), whose value fit a pre-selected range of values, which correspond so skin pixels.

In a correlation unit 173 a spatial correlation is performed of adjacent pixels or areas of pixels in the thermal images and RGB images, selected in the first and second selection units 171, 172, to detect (define) a region of interest (ROI) as skin area. Only those adjacent pixels, which correspond to the criteria of both thermal and RGB sensors, will be regarded as skin areas. From this (these) skin area(s) vital signs can then be derived by the vital signs detection unit 18 using the rPPG technology.

In a further embodiment of the invention, RGB images are processed by a PPG extraction algorithm to select spatial blocks with temporal signals, which satisfy the desired properties of PPG signals. In particular, the RGB images are divided into blocks in a block division unit 174 and PPG signals are extracted from the blocks by a block-based PPG extraction unit 175. A third selection unit 176 selects spatial blocks with the strongest PPG signal strength. In this embodiment the correlation unit 173 is configured to perform a spatial correlation of adjacent pixels or areas of pixels in the thermal images and RGB images as well as the pixels selected by the third selection unit 176 to detect (define) a ROI as skin area.

In another embodiment of the present invention, the combined thermal and RGB imaging is applied to select areas, which might have strong PPG signals. Those areas correspond to some pixels of thermal images with higher values. However, not all “high temperature” pixels correspond to locations with strong extracted PPG signals. For instance, thermal pixels located close to eyes and mouth have higher amplitudes. Although, lips of the mouth would have indeed high blood perfusion, pulsatility of a PPG signal would not be high. PPG signals extracted from skin areas around eye and mouth will be very noisy and not reliable. At the same time, PPG signals extracted from cheeks would be strong due to uniformity of a skin area.

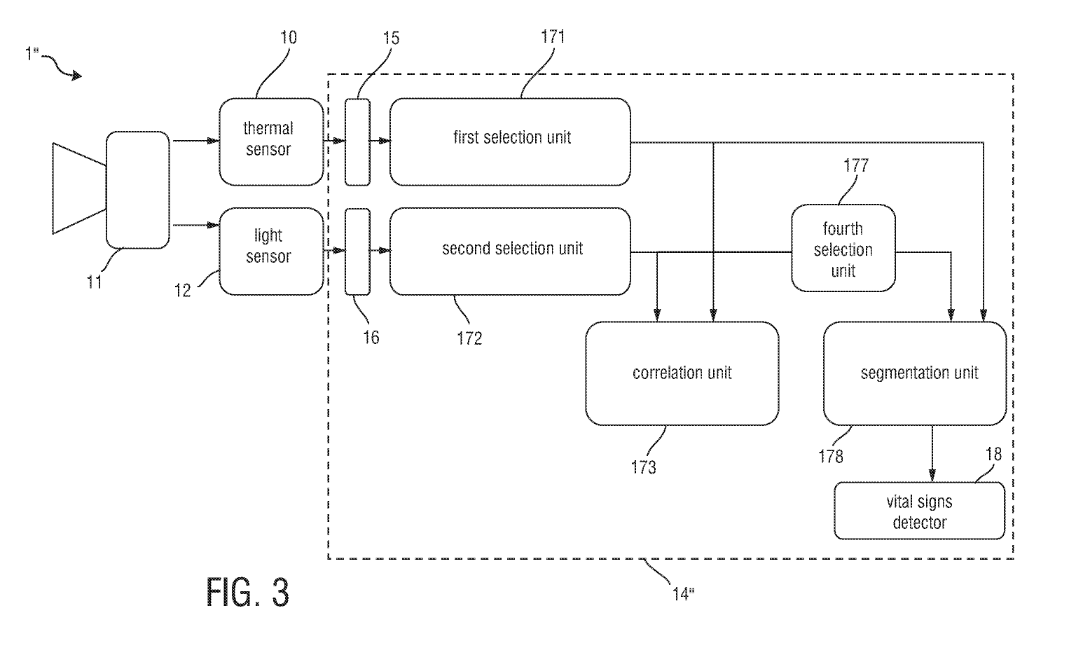

Therefore, in another embodiment of the proposed system 1″ and device 14″ as schematically shown in FIG. 3 blocks of pixels selected from a thermal image are preferably compared with pixels selected from an RGB image in the correlation unit 173. Adjacent RGB pixels are selected in a fourth selection unit 177 as candidates based on their spatial uniformity in chrominance and/or intensity, preferably in both chrominance and intensity. The fourth selection unit 177 particularly detects segments with spatially uniform values of pixels in chrominance components (for RGB) and/or in intensity (for IR sensors). In this way, pixels around mouth, eyes, hair will be rejected from the ROI with expected strong PPG signals in a segmentation unit 178.

In yet another embodiment of the present invention, thermal and RGB sensors do not share the same optical path. In this case, the alignment of images from thermal and RGB sensors may be performed digitally using image processing techniques. This embodiment allows building of a vital signs camera system by adding an extension unit with a thermal camera to an existing camera device used conventionally for acquiring RGB images and deriving vital signs there from.

The invention is preferably applied in the field of rPPG for the acquisition of vital signs of the person. Thus, images obtained by an imaging unit (used as light sensor) are not only used for detecting skin areas as explained above, but from detected (and preferably tracked, also by use of the present invention) skin areas PPG signals are derived, which are used for deriving vital signs of the person, such as heartbeat, SpO2, etc. The light sensor 12 is at least sensitive at the wavelength(s) or wavelength ranges, in which the scene is illuminated (by ambient light and/or by illumination), but may be sensitive for other wavelengths as well, in particular if required for obtaining the desired vital signs. Further, the evaluation unit 17 may be configured to select the appropriate wavelengths from the light sensor data to detect skin area(s) and, if desired, to derive vital sign(s) from the detected skin area(s) using the rPPG technology and/or by analyzing the periodic motion to obtain breathing information such as the breathing rate.

In another embodiment of the present invention, the proposed analysis for skin detection can be combined with another method for skin detection, e.g. the analysis of chrominance or temporal pulsatility of structured light reflected from the skin area as generally known.

FIG. 4 shows a flow chart of an embodiment of a method for skin detection according to the present invention. In a first step S10 thermal sensor data of a scene are obtained. In a second step S12 (which is preferably simultaneously to the first step S10) light sensor data of the scene are obtained. In a third step S14 the obtained thermal sensor data and the obtained light sensor data are analyzed. Finally, in a fourth step S16 skin areas are detected within the scene based on said analysis. Optionally, in a fifth step S18 vital signs of a subject are detected within the scene based on image information from detected skin areas within a sequence of images (representing the light sensor data). The method may comprise further steps and may be modified as explained above for the various embodiments of the device and as disclosed herein.

The proposed device and method can be used for continuous unobtrusive monitoring of PPG related vital signs (e.g. heartbeat, SpO2, respiration), and can be used in NICU, Operation Room, or General Ward. The proposed device and method can be also used for personal health monitoring. Generally, the present invention can be used in all applications where skin needs to be detected in an image of a scene and needs particularly be distinguished from non-skin.

Furthermore, the different embodiments can take the form of a computer program product accessible from a computer usable or computer readable medium providing program code for use by or in connection with a computer or any device or system that executes instructions. For the purposes of this disclosure, a computer usable or computer readable medium can generally be any tangible device or apparatus that can contain, store, communicate, propagate, or transport the program for use by or in connection with the instruction execution device.

In so far as embodiments of the disclosure have been described as being implemented, at least in part, by software-controlled data processing devices, it will be appreciated that the non-transitory machine-readable medium carrying such software, such as an optical disk, a magnetic disk, semiconductor memory or the like, is also considered to represent an embodiment of the present disclosure.

The computer usable or computer readable medium can be, for example, without limitation, an electronic, magnetic, optical, electromagnetic, infrared, or semiconductor system, or a propagation medium. Non-limiting examples of a computer readable medium include a semiconductor or solid state memory, magnetic tape, a removable computer diskette, a random access memory (RAM), a read-only memory (ROM), a rigid magnetic disk, and an optical disk. Optical disks may include compact disk-read only memory (CD-ROM), compact disk-read/write (CD-R/W), and DVD.

Further, a computer usable or computer readable medium may contain or store a computer readable or usable program code such that when the computer readable or usable program code is executed on a computer, the execution of this computer readable or usable program code causes the computer to transmit another computer readable or usable program code over a communications link. This communications link may use a medium that is, for example, without limitation, physical or wireless.

A data processing system or device suitable for storing and/or executing computer readable or computer usable program code will include one or more processors coupled directly or indirectly to memory elements through a communications fabric, such as a system bus. The memory elements may include local memory employed during actual execution of the program code, bulk storage, and cache memories, which provide temporary storage of at least some computer readable or computer usable program code to reduce the number of times code may be retrieved from bulk storage during execution of the code.

Input/output, or I/O devices, can be coupled to the system either directly or through intervening I/O controllers. These devices may include, for example, without limitation, keyboards, touch screen displays, and pointing devices. Different communications adapters may also be coupled to the system to enable the data processing system to become coupled to other data processing systems, remote printers, or storage devices through intervening private or public networks. Non-limiting examples are modems and network adapters and are just a few of the currently available types of communications adapters.

The description of the different illustrative embodiments has been presented for purposes of illustration and description and is not intended to be exhaustive or limited to the embodiments in the form disclosed. Many modifications and variations will be apparent to those of ordinary skill in the art. Further, different illustrative embodiments may provide different advantages as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

1. A device for skin detection comprising:
a thermal sensor input configured to obtain thermal sensor data of a scene,
a light sensor input configured to obtain light sensor data of the scene, and
an evaluation unit configured to analyze the obtained thermal sensor data and the obtained light sensor data and to detect skin areas within the scene based on said analysis, wherein said evaluation unit is configured
to select first spatial areas within the thermal sensor data having data values in a predetermined range,
to select second spatial areas within the light sensor data having data values in a predetermined range,
to select third and/or fourth spatial areas by determining photoplethysmography, PPG, signals from the light sensor data and selecting third and/or fourth spatial areas within the light sensor data having the highest PPG signal strengths and/or having the strongest pulsatility, and/or to select fifth spatial areas within the light sensor data having the highest spatial uniformity of chrominance and/or intensity values of adjacent pixels, and
to correlate said first and second spatial areas with one or more of said third, fourth and fifth spatial areas to detect skin areas.

2. The device as claimed in claim 1,
wherein said evaluation unit is configured to select first spatial areas within the thermal sensor data having data values in a range of normal temperature of a human skin and to select second spatial areas within the light sensor data having data values in a range typical for skin.

3. The device as claimed in claim 2,
wherein said evaluation unit is configured to select first spatial areas within the thermal sensor data having data values in a range of 30 to 42° C.

4. The device as claimed in claim 1,
further comprising a vital signs detector configured to detect vital signs of a subject within the scene based on image information from detected skin areas within said sequence of images.

5. The device as claimed in claim 1,
wherein said evaluation unit is configured to spatially align the obtained thermal sensor data and the obtained light sensor data before the analysis.

6. A system for skin detection comprising:
a thermal sensor configured to acquire thermal sensor data of a scene,
a light sensor configured to acquire light sensor data of the scene, and
a device as claimed in claim 1 for skin detection within the scene based on the acquired thermal sensor data and the acquired light sensor data.

7. The system as claimed in claim 6,
wherein said thermal sensor comprises a longwave camera unit configured to acquire thermal images in the longwave infrared spectrum.

8. The system as claimed in claim 6,
wherein said light sensor comprises an imaging unit configured to acquire images in the visible and/or infrared light spectrum.

9. The system as claimed in claim 6,
wherein said thermal sensor and said light sensor are arranged to have substantially the same optical path

10. A method for skin detection comprising:
obtaining thermal sensor data of a scene,
obtaining light sensor data of the scene,
analyzing the obtained thermal sensor data and the obtained light sensor data by
selecting first spatial areas within the thermal sensor data having data values in a predetermined range,
selecting second spatial areas within the light sensor data having data values in a predetermined range, and
selecting third and/or fourth spatial areas by determining photoplethysmography, PPG, signals from the light sensor data and selecting third and/or fourth spatial areas within the light sensor data having the highest PPG signal strengths and/or having the strongest pulsatility, and/or to select fifth spatial areas within the light sensor data having the highest spatial uniformity of chrominance and/or intensity values of adjacent pixels, and
detecting skin areas within the scene based on said analysis by correlating said first and second spatial areas with one or more of said third, fourth and fifth spatial areas.

11. A computer readable non-transitory medium having instructions stored thereon which, when carried out on a computer, cause the computer to perform the steps of the method as claimed in claim 10.
